(19) United States
(12) Patent Application Publication
(10) Pub. No.: US 2025/0049395 A1
(43) Pub. Date: Feb. 13, 2025

(54) APPARATUS, SYSTEM, AND METHOD FOR REMOTE VITAL SIGN COLLECTION

(71) Applicant: VitalitusMed LLC, West Hempstead, NY (US)

(72) Inventor: Terrance Bloom, Surrey (CA)

(73) Assignee: VitalitusMed LLC, West Hempstead, NY (US)

(21) Appl. No.: 18/232,817

(22) Filed: Aug. 10, 2023

(51) Int. Cl. A61B 5/00; A61B 5/024

(52) U.S. Cl. CPC A61B 5/6833; A61B 5/02438; A61B 5/7282; A61B 5/746; A61B 2562/0219; A61B 2562/166; A61B 2562/242

(57) ABSTRACT

A wireless vital sign patch apparatus comprising a printed circuit board (“PCB”) comprising a transmitter; a plurality of sensors comprising a heartrate monitor, a gyroscope, and an accelerometer. The wireless vital sign patch apparatus may comprise at least one processor, at least one memory comprising computer-executable instructions which, when executed by the at least one processor, cause the at least one processor to package data to be transmitted, via the transmitter, to an external device based on a criteria; and a housing encapsulating the PCB, the housing composed a flexible material, the housing comprising an interfacing surface configured to reversibly interface with a patient.

### FIELD OF THE DISCLOSURE

The present disclosure is generally related to vital sign monitoring, and more particularly, to an apparatus, system, and method for remote vital sign collection.

### INTRODUCTION

Vital readings are often taken by wired devices. These wired devices restrict patient range of motion and may even present a tripping hazard. Some wireless vital collection devices exist, but are rigid, have limited areas in which they can be applied, and are costly to manufacture. Further, such wireless devices are often cumbersome and/or difficult to affix to a patient.

Therefore, there is a need for an improved system and method for obtaining vital signs and manufacturing patches capable of obtaining said vital signs that could overcome the aforementioned issues. It would further be desirable to have an improved system and method for creating patches for use upon a desired thoracic location.

### SUMMARY OF THE INVENTION

The invention of the present disclosure may be a wireless vital sign patch apparatus comprising a printed circuit board (“PCB”) comprising a transmitter and a plurality of sensors comprising a heartrate monitor, a gyroscope, and an accelerometer. The PCB may comprise at least one processor, at least one memory comprising computer-executable instructions which, when executed by the at least one processor, cause the at least one processor to package data to be transmitted, via the transmitter, to an external device based on a criteria. The apparatus may include a housing encapsulating the PCB, the housing composed a flexible material, the housing comprising an interfacing surface configured to reversibly interface with a patient.

The flexible material may be transparent. The flexible material may be silicone. In various embodiments, the housing is impregnated with an antimicrobial agent, an anesthetic agent, and/or a prescribed drug.

The housing may further comprise an adhesive disposed on the interfacing surface. In another embodiment, the flexible material includes an acoustic impedance similar to that of human tissue.

The invention of the present disclosure may be a wireless vital sign detection system comprising a patch comprising a printed circuit board (“PCB”) comprising a transmitter and a plurality of sensors comprising a heartrate monitor, a gyroscope, and an accelerometer. In an embodiment, the PCB comprises at least one patch processor, at least one patch memory comprising computer-executable patch instructions which, when executed by the at least one patch processor, cause the at least one patch processor to package data to be transmitted, via the transmitter, to an external device based on a criteria. The patch may include a housing encapsulating the PCB, the housing composed a flexible material, the housing comprising an interfacing surface configured to reversibly interface with a patient. The system may further comprise an external device comprising a receiver in wireless communication with the transmitter and at least one external processor, at least one external memory comprising computer-executable external instructions which, when executed by the at least one external processor, cause the at least one external processor to receive, via the receiver, the packaged data; determine, via the at least one external processor, a clinically significant event based on a determined pathology; and provide an indication when the clinically significant event is detected.

The flexible material may be transparent. The flexible material may be silicone. In various embodiments, the housing is impregnated with an antimicrobial agent, an anesthetic agent, and/or a prescribed drug. The housing may further comprise an adhesive disposed on the interfacing surface. In another embodiment, the flexible material includes an acoustic impedance similar to that of human tissue.

It is to be understood that both the forgoing and the following descriptions are exemplary and explanatory only and are not intended to limit the claimed disclosure or application thereof in any manner whatsoever.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 is an illustrative block diagram of a system based on a computer for execution of a remote vital sign collection system.

FIG. 2 is an illustration of a computing machine for execution of a remote vital sign collection system.

FIG. 3 is a cross-sectional view of an embodiment of a patch.

FIG. 4 is an illustration of an embodiment of a patch.

FIG. 5 is an illustration of an embodiment of a patch circuit board.

### DETAILED DESCRIPTION

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

Those skilled in the art will realize that storage devices utilized to provide computer-readable and computer-executable instructions and data can be distributed over a network. For example, a remote computer or storage device may store computer-readable and computer-executable instructions in the form of software applications and data. A local computer may access the remote computer or storage device via the network and download part or all of a software application or data and may execute any computer-executable instructions. Alternatively, the local computer may download pieces of the software or data as needed or process the software in a distributive manner by executing some of the instructions at the local computer and some at remote computers and/or devices.

Those skilled in the art will also realize that, by utilizing conventional techniques, all or portions of the software's computer-executable instructions may be carried out by a dedicated electronic circuit such as a digital signal processor (“DSP”), programmable logic array (“PLA”), discrete circuits, and the like. The term “electronic apparatus” may include computing devices or consumer electronic devices comprising any software, firmware or the like, or electronic devices or circuits comprising no software, firmware or the like.

The term “firmware” as used herein typically includes and refers to executable instructions, code, data, applications, programs, program modules, or the like maintained in an electronic device such as a ROM. The term “software” as used herein typically includes and refers to computer-executable instructions, code, data, applications, programs, program modules, firmware, and the like maintained in or on any form or type of computer-readable media that is configured for storing computer-executable instructions or the like in a manner that may be accessible to a computing device.

The terms “computer-readable medium”, “computer-readable media”, and the like as used herein and in the claims are limited to referring strictly to one or more statutory apparatus, article of manufacture, or the like that is not a signal or carrier wave per se. Thus, computer-readable media, as the term is used herein, is intended to be and must be interpreted as statutory subject matter.

The term “computing device” as used herein and in the claims is limited to referring strictly to one or more statutory apparatus, article of manufacture, or the like that is not a signal or carrier wave per se, such as computing device 101 that encompasses client devices, mobile devices, wearable devices, one or more servers, network services such as an Internet services or corporate network services based on one or more computers, and the like, and/or any combination thereof. Thus, a computing device, as the term is used herein, is also intended to be and must be interpreted as statutory subject matter.

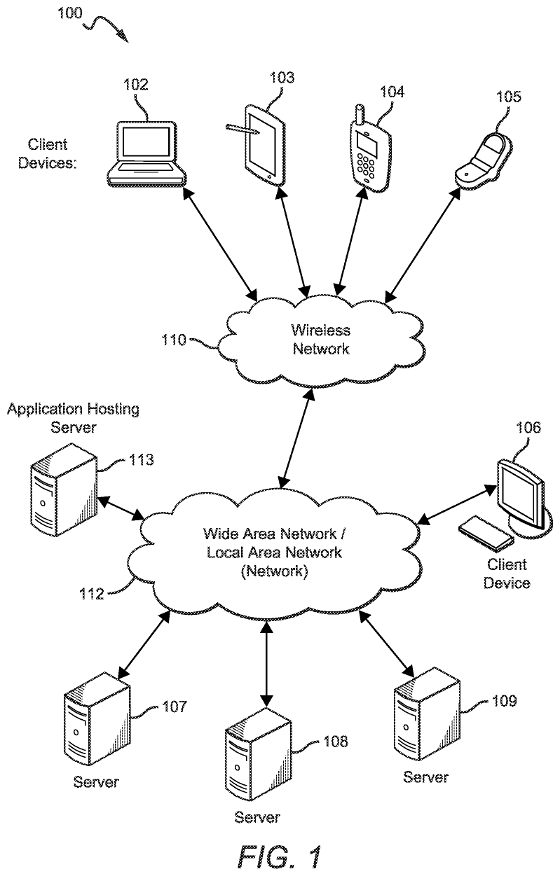

FIG. 1 illustrates components of one embodiment of an environment in which the invention may be practiced. Not all of the components may be required to practice the invention, and variations in the arrangement and type of the components may be made without departing from the spirit or scope of the invention. As shown, the system 100 includes one or more Local Area Networks (“LANs”)/Wide Area Networks (“WANs”) 112, one or more wireless networks 110, one or more wired or wireless client devices 106, mobile or other wireless client devices 102-105, servers 107-109, and may include or communicate with one or more data stores or databases. Various of the client devices 102-106 may include, for example, desktop computers, laptop computers, set top boxes, tablets, cell phones, smart phones, smart speakers, wearable devices (such as the Apple Watch) and the like. Servers 107-109 can include, for example, one or more application servers, content servers, search servers, and the like. FIG. 1 also illustrates application hosting server 113.

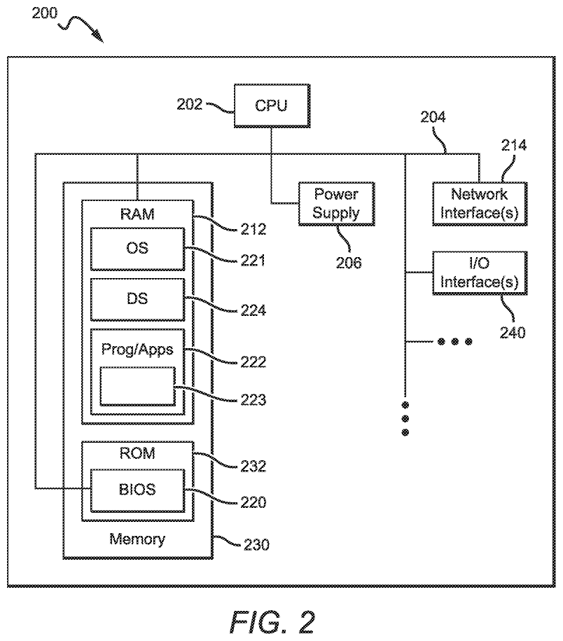

FIG. 2 illustrates a block diagram of an electronic device 200 that can implement one or more aspects of an apparatus, system and method for validating and correcting user information (the “Engine”) according to one embodiment of the invention. Instances of the electronic device 200 may include servers, e.g., servers 107-109, and client devices, e.g., client devices 102-106. In general, the electronic device 200 can include a processor/CPU 202, memory 230, a power supply 206, and input/output (I/O) components/devices 240, e.g., microphones, speakers, displays, touchscreens, keyboards, mice, keypads, microscopes, GPS components, cameras, heart rate sensors, light sensors, accelerometers, targeted biometric sensors, etc., which may be operable, for example, to provide graphical user interfaces or text user interfaces.

A user may provide input via a touchscreen of an electronic device 200. A touchscreen may determine whether a user is providing input by, for example, determining whether the user is touching the touchscreen with a part of the user's body such as his or her fingers. The electronic device 200 can also include a communications bus 204 that connects the aforementioned elements of the electronic device 200. Network interfaces 214 can include a receiver and a transmitter (or transceiver), and one or more antennas for wireless communications.

The processor 202 can include one or more of any type of processing device, e.g., a Central Processing Unit (CPU), and a Graphics Processing Unit (GPU). Also, for example, the processor can be central processing logic, or other logic, may include hardware, firmware, software, or combinations thereof, to perform one or more functions or actions, or to cause one or more functions or actions from one or more other components. Also, based on a desired application or need, central processing logic, or other logic, may include, for example, a software-controlled microprocessor, discrete logic, e.g., an Application Specific Integrated Circuit (ASIC), a programmable/programmed logic device, memory device containing instructions, etc., or combinatorial logic embodied in hardware. Furthermore, logic may also be fully embodied as software.

The memory 230, which can include Random Access Memory (RAM) 212 and Read Only Memory (ROM) 232, can be enabled by one or more of any type of memory device, e.g., a primary (directly accessible by the CPU) or secondary (indirectly accessible by the CPU) storage device (e.g., flash memory, magnetic disk, optical disk, and the like). The RAM can include an operating system 221, data storage 224, which may include one or more databases, and programs and/or applications 222, which can include, for example, software aspects of the program 223. The ROM 232 can also include Basic Input/Output System (BIOS) 220 of the electronic device.

Software aspects of the program 223 are intended to broadly include or represent all programming, applications, algorithms, models, software and other tools necessary to implement or facilitate methods and systems according to embodiments of the invention. The elements may exist on a single computer or be distributed among multiple computers, servers, devices or entities.

The power supply 206 contains one or more power components and facilitates supply and management of power to the electronic device 200.

The input/output components, including Input/Output (I/O) interfaces 240, can include, for example, any interfaces for facilitating communication between any components of the electronic device 200, components of external devices (e.g., components of other devices of the network or system 100), and end users. For example, such components can include a network card that may be an integration of a receiver, a transmitter, a transceiver, and one or more input/output interfaces. A network card, for example, can facilitate wired or wireless communication with other devices of a network. In cases of wireless communication, an antenna can facilitate such communication. Also, some of the input/output interfaces 240 and the bus 204 can facilitate communication between components of the electronic device 200, and in an example can ease processing performed by the processor 202.

Where the electronic device 200 is a server, it can include a computing device that can be capable of sending or receiving signals, e.g., via a wired or wireless network, or may be capable of processing or storing signals, e.g., in memory as physical memory states. The server may be an application server that includes a configuration to provide one or more applications, e.g., aspects of the Engine, via a network to another device. Also, an application server may, for example, host a web site that can provide a user interface for administration of example aspects of the Engine.

Any computing device capable of sending, receiving, and processing data over a wired and/or a wireless network may act as a server, such as in facilitating aspects of implementations of the Engine. Thus, devices acting as a server may include devices such as dedicated rack-mounted servers, desktop computers, laptop computers, set top boxes, integrated devices combining one or more of the preceding devices, and the like.

Servers may vary widely in configuration and capabilities, but they generally include one or more central processing units, memory, mass data storage, a power supply, wired or wireless network interfaces, input/output interfaces, and an operating system such as Windows Server, Mac OS X, Unix, Linux, FreeBSD, and the like.

A server may include, for example, a device that is configured, or includes a configuration, to provide data or content via one or more networks to another device, such as in facilitating aspects of an example apparatus, system and method of the Engine. One or more servers may, for example, be used in hosting a Web site, such as the web site www.microsoft.com. One or more servers may host a variety of sites, such as, for example, business sites, informational sites, social networking sites, educational sites, wikis, financial sites, government sites, personal sites, and the like.

Servers may also, for example, provide a variety of services, such as Web services, third-party services, audio services, video services, email services, HTTP or HTTPS services, Instant Messaging (IM) services, Short Message Service (SMS) services, Multimedia Messaging Service (MMS) services, File Transfer Protocol (FTP) services, Voice Over IP (VOIP) services, calendaring services, phone services, and the like, all of which may work in conjunction with example aspects of an example systems and methods for the apparatus, system and method embodying the Engine. Content may include, for example, text, images, audio, video, and the like.

In example aspects of the apparatus, system and method embodying the Engine, client devices may include, for example, any computing device capable of sending and receiving data over a wired and/or a wireless network. Such client devices may include desktop computers as well as portable devices such as cellular telephones, smart phones, display pagers, Radio Frequency (RF) devices, Infrared (IR) devices, Personal Digital Assistants (PDAs), handheld computers, GPS-enabled devices tablet computers, sensor-equipped devices, laptop computers, set top boxes, wearable computers such as the Apple Watch and Fitbit, integrated devices combining one or more of the preceding devices, and the like.

Client devices such as client devices 102-106, as may be used in an example apparatus, system and method embodying the Engine, may range widely in terms of capabilities and features. For example, a cell phone, smart phone or tablet may have a numeric keypad and a few lines of monochrome Liquid-Crystal Display (LCD) display on which only text may be displayed. In another example, a Web-enabled client device may have a physical or virtual keyboard, data storage (such as flash memory or SD cards), accelerometers, gyroscopes, respiration sensors, body movement sensors, proximity sensors, motion sensors, ambient light sensors, moisture sensors, temperature sensors, compass, barometer, fingerprint sensor, face identification sensor using the camera, pulse sensors, heart rate variability (HRV) sensors, beats per minute (BPM) heart rate sensors, microphones (sound sensors), speakers, GPS or other location-aware capability, and a 2D or 3D touch-sensitive color screen on which both text and graphics may be displayed. In some embodiments multiple client devices may be used to collect a combination of data. For example, a smart phone may be used to collect movement data via an accelerometer and/or gyroscope and a smart watch (such as the Apple Watch) may be used to collect heart rate data. The multiple client devices (such as a smart phone and a smart watch) may be communicatively coupled.

Client devices, such as client devices 102-106, for example, as may be used in an example apparatus, system and method implementing the Engine, may run a variety of operating systems, including personal computer operating systems such as Windows, iOS or Linux, and mobile operating systems such as iOS, Android, Windows Mobile, and the like. Client devices may be used to run one or more applications that are configured to send or receive data from another computing device. Client applications may provide and receive textual content, multimedia information, and the like. Client applications may perform actions such as browsing webpages, using a web search engine, interacting with various apps stored on a smart phone, sending and receiving messages via email, SMS, or MMS, playing games (such as fantasy sports leagues), receiving advertising, watching locally stored or streamed video, or participating in social networks.

In example aspects of the apparatus, system and method implementing the Engine, one or more networks, such as networks 110 or 112, for example, may couple servers and client devices with other computing devices, including through wireless network to client devices. A network may be enabled to employ any form of computer readable media for communicating information from one electronic device to another. The computer readable media may be non-transitory. A network may include the Internet in addition to Local Area Networks (LANs), Wide Area Networks (WANs), direct connections, such as through a Universal Serial Bus (USB) port, other forms of computer-readable media (computer-readable memories), or any combination thereof. On an interconnected set of LANs, including those based on differing architectures and protocols, a router acts as a link between LANs, enabling data to be sent from one to another.

Communication links within LANs may include twisted wire pair or coaxial cable, while communication links between networks may utilize analog telephone lines, cable lines, optical lines, full or fractional dedicated digital lines including T1, T2, T3, and T4, Integrated Services Digital Networks (ISDNs), Digital Subscriber Lines (DSLs), wireless links including satellite links, optic fiber links, or other communications links known to those skilled in the art. Furthermore, remote computers and other related electronic devices could be remotely connected to either LANs or WANs via a modem and a telephone link.

A wireless network, such as wireless network 110, as in an example apparatus, system and method implementing the Engine, may couple devices with a network. A wireless network may employ stand-alone ad-hoc networks, mesh networks, Wireless LAN (WLAN) networks, cellular networks, and the like.

A wireless network may further include an autonomous system of terminals, gateways, routers, or the like connected by wireless radio links, or the like. These connectors may be configured to move freely and randomly and organize themselves arbitrarily, such that the topology of wireless network may change rapidly. A wireless network may further employ a plurality of access technologies including 2nd (2G), 3rd (3G), 4th (4G) generation, Long Term Evolution (LTE) radio access for cellular systems, WLAN, Wireless Router (WR) mesh, and the like. Access technologies such as 2G, 2.5G, 3G, 4G, and future access networks may enable wide area coverage for client devices, such as client devices with various degrees of mobility. For example, a wireless network may enable a radio connection through a radio network access technology such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, and the like. A wireless network may include virtually any wireless communication mechanism by which information may travel between client devices and another computing device, network, and the like.

Internet Protocol (IP) may be used for transmitting data communication packets over a network of participating digital communication networks, and may include protocols such as TCP/IP, UDP, DECnet, NetBEUI, IPX, Appletalk, and the like. Versions of the Internet Protocol include IPv4 and IPv6. The Internet includes local area networks (LANs), Wide Area Networks (WANs), wireless networks, and long-haul public networks that may allow packets to be communicated between the local area networks. The packets may be transmitted between nodes in the network to sites each of which has a unique local network address. A data communication packet may be sent through the Internet from a user site via an access node connected to the Internet. The packet may be forwarded through the network nodes to any target site connected to the network provided that the site address of the target site is included in a header of the packet. Each packet communicated over the Internet may be routed via a path determined by gateways and servers that switch the packet according to the target address and the availability of a network path to connect to the target site.

The header of the packet may include, for example, the source port (16 bits), destination port (16 bits), sequence number (32 bits), acknowledgement number (32 bits), data offset (4 bits), reserved (6 bits), checksum (16 bits), urgent pointer (16 bits), options (variable number of bits in multiple of 8 bits in length), padding (may be composed of all zeros and includes a number of bits such that the header ends on a 32 bit boundary). The number of bits for each of the above may also be higher or lower.

A “content delivery network” or “content distribution network” (CDN), as may be used in an example apparatus, system and method implementing the Engine, generally refers to a distributed computer system that comprises a collection of autonomous computers linked by a network or networks, together with the software, systems, protocols and techniques designed to facilitate various services, such as the storage, caching, or transmission of content, streaming media and applications on behalf of content providers. Such services may make use of ancillary technologies including, but not limited to, “cloud computing,” distributed storage, DNS request handling, provisioning, data monitoring and reporting, content targeting, personalization, and business intelligence. A CDN may also enable an entity to operate and/or manage a third party's web site infrastructure, in whole or in part, on the third party's behalf.

A Peer-to-Peer (or P2P) computer network relies primarily on the computing power and bandwidth of the participants in the network rather than concentrating it in a given set of dedicated servers. P2P networks are typically used for connecting nodes via largely ad hoc connections. A pure peer-to-peer network does not have a notion of clients or servers, but only equal peer nodes that simultaneously function as both “clients” and “servers” to the other nodes on the network.

Embodiments of the present invention include apparatuses, systems, and methods implementing the Engine. Embodiments of the present invention may be implemented on one or more of client devices 102-106, which are communicatively coupled to servers including servers 107-109. Moreover, client devices 102-106 may be communicatively (wirelessly or wired) coupled to one another. In particular, software aspects of the Engine may be implemented in the program 223. The program 223 may be implemented on one or more client devices 102-106, one or more servers 107-109, and 113, or a combination of one or more client devices 102-106, and one or more servers 107-109 and 113.

In an embodiment, the system may receive, process, generate and/or store time series data. The system may include an application programming interface (API). The API may include an API subsystem. The API subsystem may allow a data source to access data. The API subsystem may allow a third-party data source to send the data. In one example, the third-party data source may send JavaScript Object Notation (“JSON”)-encoded object data. In an embodiment, the object data may be encoded as XML-encoded object data, query parameter encoded object data, or byte-encoded object data.

The invention of the present disclosure may be a patch, for example a physiological biosensor. The patch may be a disposable single-use remote wearable biosensor that may be used by inpatient and outpatient service providers, clinical research departments and/or individual end users to monitor and detect a number of variables and vital signs. As non-limiting examples, the patch may detect and monitor body temperature, heart rate, body position, and SpO₂ (i.e., oxygen saturation). In an embodiment, the patch utilizes Bluetooth communication capabilities to transmit and store data to an online proprietary platform that may translate raw data into an easy and accessible output. The patch may not be restricted to limited placement of the device on particular parts of the body (for example, on the wrist), but rather the patch may be placed at any thoracic location of the body all while continuing to provide consistent monitoring results. In an embodiment, the patch may also include a microphone (for example, to analyze cardiovascular noises, respiratory auscultation, palpitations, ambient noise, speech, requests for help, etc.).

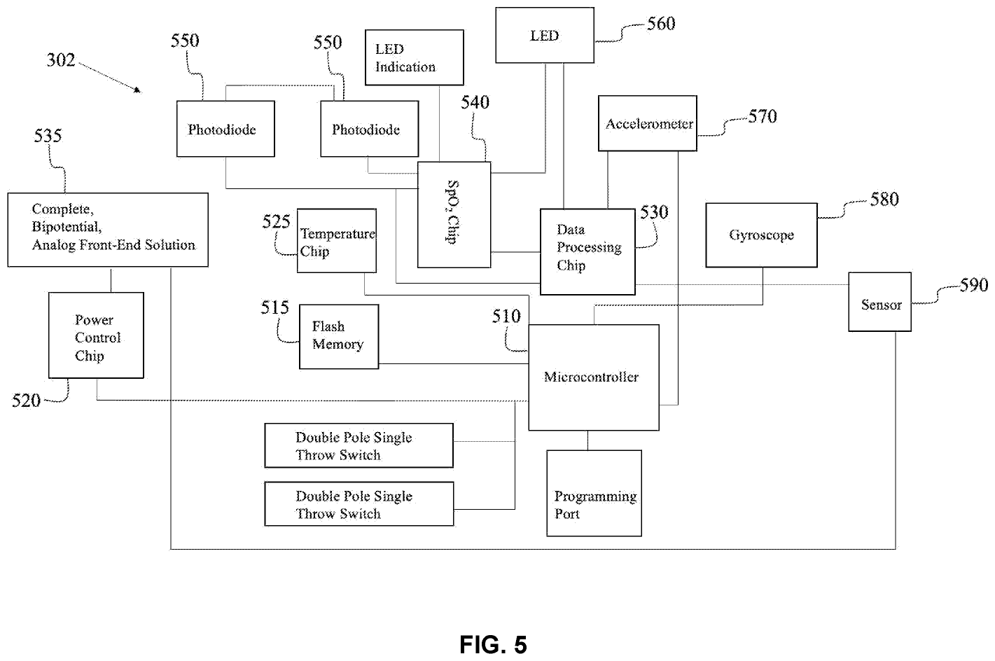

In an embodiment, the patch may include a Bluetooth chip, a memory module capable of storing computer executable instructions, a power supply, a battery, a wireless charger, a SpO₂ chip 540, one or more LEDs 560, an EKG/ECG lead/sensor, a temperature chip 525, an accelerometer, a gyroscope 580, a data processing chip 530, a photodiode 550, a heartrate detector, a pedometer, and/or a microphone. Accordingly, these aforementioned components may include the features and technical aspects of the components described above and displayed in FIGS. 1 and 2. However, the patch may include any number or combination of components. Referring to FIG. 5, in an embodiment, the patch includes a microcontroller 510 (i.e., NRF52832), where the microcontroller 510 is a central processing unit configured to control the communication with and between the other components and/or sensors.

In an embodiment, a power control chip 520 (i.e., MAX20303) may enable the power supply to deliver power to the components on the board. A Bluetooth chip may send the data over Bluetooth to a nearby mobile device. However, the Bluetooth chip may be supplemented and/or replaced with any component configured to transmit data. For example, the patch may include any component adapted to transmit data via a short-range wireless technology or any suitable radio waves. The mobile device may include an “app” configured to upload the data to a server, where the data may be processed and/or displayed. The signal may also be sent to a base station, which may transmit the signal through an electrically or communicatively connected server. The signal may be transmitted by a variety of different methods. The signal may be encrypted, modulated, or altered in a variety of manners to allow for security, privacy, or data compression. For example, the signal may be encrypted using a cryptographic hash function to ensure secure transmission or other cryptographic functions to comply with HIPAA or similar regulation. In another example, the signal may be dynamically compressed to different ratios to allow for consistent health information updates. For example, as the signal strength changes, the update/refresh rate of the device may be dynamically increased or decreased. In a further example, certain vital signals may be assigned different priorities, so if bandwidth is constrained then the device may not update the SpO₂ value as often and prioritize monitoring the patient's heartrate. In an embodiment, the patch may contain a processor that processes the signal to be sent to another device to select a subset of the data to be transmitted, this subset may be selected for using a variety of factors such as data quality, relevance, noise, etc. In an embodiment, the device may be configured to a particular patient profile, for example, a patient may have a preliminary diagnosis of pneumonia, therefore the device may be configured to run a particular pathology profile where SpO₂, heartrate, and microphone data are prioritized, and body temperature and gyroscopic data are deprioritized. These profiles may contain any permutation of any sensor data. The device may be configured to accept custom pathology profiles, have pathology profiles selected, or may only contain a subset of only the relevant sensor for a particular pathology in order to achieve cost efficiency standards. The pathology profiles may monitor for certain clinical events. For example, normally a patient SpO₂ value may be determined to be acceptable if greater than 90%, however if a patient has pneumonia, the acceptable value may be higher or lower, and if this set threshold value is crossed, an alert or indication may be triggered and/or transmitted. In another embodiment, the Bluetooth chip also includes Near Field Communication (“NFC”) for identifying each board and/or board component.

The data processing chip 530 (i.e., MAX32664C) may communicate with the SpO₂ chip 540 (i.e., MAX86141), the analog multiplexer (i.e., MAX4740ETE+), the photodiode 550 (i.e., VEMD8080), and/or one or more LEDs 560 (i.e., FIREFLYE2218 and/or SFH7013). In an embodiment, the data processing chip 530 processes the data from the SpO₂ chip 540, accelerometer 570, and/or gyroscope 580, where the SpO₂ chip 540, in conjunction with the analog multiplexer, photodiode 550, and/or LEDs 560, may generate and display Heart Rate and SpO₂ readings. Further, the processing chip 530 may utilize the accelerometer 570 and/or gyroscope 580 to generate and/or display the activity of patient. In an embodiment, the sensors 590 and/or other patch components may be used to detect when an adverse event occurs. For example, if the accelerometer 570 detects a particular acceleration of more than a determined magnitude for a set duration, the processor may determine the patient has been in an accident or fallen, after which the patch may send this information to a third party.

The SpO₂ chip 540 may drive the LEDs 560 and may interpret and/or, otherwise receive, reflected light data from the photodiodes 550. Further, this data may be sent to the data processing chip 530 to determine heart rate and/or SpO₂.

The accelerometer 570 and/or gyroscope 580 (i.e., configured to collect data related to physical movements) may gather data that is next transmitted to the data processing chip 530 for processing to determine the activity of the patient (i.e., a state of the patient, such as sleeping, resting, walking, running, or biking).

The analog multiplexer may be configured to control 2 LEDs via the SpO₂ chip 540. In an embodiment, the patch includes a series of LEDs 560, each configured to emit a different frequency of light (i.e., red, green and infrared). In an embodiment, one of the one or more LEDs 560 may emit a single color of light (i.e., green). However, the LEDs 560 may be adapted to display any color, pattern, or duration of light as a function of a condition determined by the processing chip 530.

In an embodiment, the photodiode 550 is configured to read a reflected light amount and may further be interpreted by the data processing chip 530. The patch may include a flash memory 515 (i.e., MX25U51245G of size 64 Mega Bytes), to store patient data with time stamps to be recovered later by a doctor or clinician. Accordingly, such a doctor may be permitted access to a particular patient's parameters at any time. On board memory may be used when the signal strength/bandwidth with the external device is limited. The on board memory may be utilized as a buffer for data to be sent to an external device.

In an embodiment, the patch may include a complete, biopotential, analog front-end solution 535 (i.e., MAX3003CTI) for wearable applications. The analog front end 535 may offer high performance for clinical and fitness applications, with ultra-low power for long battery life. The analog front end 535 may include a single biopotential channel providing electrocardiogram (“ECG”) waveforms and heart rate detection.

In an embodiment, the patch includes a digital microphone (i.e., MP34DB02) to monitor breathing or, for example, other sounds from the chest cavity. The patch may include a temperature chip 525 (i.e., MAX30208E) configured to detect a patient's body temperature.

In an embodiment, the patch may include an array of microphones to measure the difference in time between detecting a sound, and use the determined differential to determine a direction of the sound in 2D or 3D space. The patch may use various signal processing methods, noise isolation, and/or noise cancellation to filter out speech, unwanted ambient noise, etc. The microphones may receive various frequencies, including those not audible to humans. In some embodiments the patch may have an ultrasonic emitter and a microphone configured to receive the ultrasonic emission. The patch housing may be composed of an ultrasound compatible material, such as ultrasound gel or material with an acoustic impedance similar to human tissue, or may have multiple layers, where one slowly melts to ensure a clear image of the anatomy being imaged. The sonic emitter may be in an array of multiple emitters, wherein the emitter array uses constructive and destructive interference to electronically steer the emitted beam wavefront. The processor on the patch may process the signal or may transmit the signal to another device for processing. The patch may be used in conjunction with another patch with another ultrasound array and the signal propagation in the thoracic anatomy to image alternative views that may not be resolvable with a single emitter location for an anatomy of interest. The additional patch may communicate with the first patch to coordinate the electronic steering of the beam wavefront or to increase the received signal to increase resolution of the anatomy of interest.

The ultrasonic array may be a variety of 2D or 3D shapes. They may be arranged in a curve or parabolic arc, however, they may be arranged in a dome, hemisphere, or parabolic dish shape. The receiver or receivers may be placed among the emitters, in front of, or behind the emitters. The receiver may be placed at a focal point of the shape. The emitter and receiver may be separate units or a single unit, they may also be transducers.

In some embodiments, the housing may be divided into subsections each capable of receiving a signal. The differences between when the signals are detected by the subsections may be used to determine location and process the signal to reduce noise, increase signal strength, etc. This, electronic steering of signals, and similar methods may allow the device to be placed on a much larger area of the torso. The device may also be placed underneath a supine patient, wherein the housing is soft and/or a flexible gel.

In some embodiments, the patch may be constructed from a printed circuit board (“PCB”) placed into a mold filled with a liquid such as silicone for efficient manufacturing. The mold may aid in consistent manufacturing and allows the patch tolerances to be tightly controlled to enable the use of sensors that would not normally work in silicone or other fluids. The silicone may be opaque or transparent. In some embodiments, highly transparent silicone may be used for uninterrupted use of the LED 560, sound emitter, and/or microphone. The housing may be shaped akin to a “suction cup,” for example, having a concave bottom surface, as to promote positive attachment to the patient. In another embodiment, the housing may include one or more adhesive layers or coatings such that the housing may be reversibly adhered to a patient.

In some embodiments, the patch may be distributed as a kit to be assembled at a desired location. The kit may contain packaging that may also contain the mold shape, a premeasured amount of the housing to be poured into the mold, and the PCB with sensors. The PCB may include a port or may use the transceiver to be configured to a pathology profile before or after pouring the housing.

In an embodiment, each of the patch components is disposed on the PCB. In an embodiment, the PCB is encapsulated by a housing. The housing may be configured to interface with a patient's skin. The housing may be a sealed silicone cover.

In an embodiment, the housing may be composed of several different layers. The patch may contain a battery, a wireless charger, a solar panel, or various other power supplies. The housing may be made of a material that reacts with the patient's body fluids or skin to produce an electrochemical gradient capable of powering the device. The housing may also be and/or may function as a battery. For example, the housing may be or may include an anode and a cathode separated by a separator and/or electrolyte. In some embodiments, the patch may be disposable for sanitary reasons. The housing, wherein the housing includes battery functionality, may be activated by damaging the separator or activating the electrolyte separating the housing layers. This damage or activation may be accomplished through mechanical means such as shaking, bending, piercing, or may be accomplished through chemical means, such as, application of heat or electrical current.

The housing may be impregnated with various chemical compounds. The housing may contain antimicrobial agents, hemostatic agents, pharmaceutical agents, anesthetic agents, or prescribed drugs. The housing may also be impregnated with an abrasive agent that may aid in facilitating transdermal absorption of any desired agent. Further, because the agents may be suspended in the housing, with limited exposure to atmospheric conditions, the housing may use compounds that are normally too volatile or reactive to allow transdermal administration.

The data collected by the patch may be transmitted to a mobile device or other electronic device (e.g., a laptop computer or tablet). Further, the data may be transmitted from the mobile device to a server. The server may then transmit the data to a website, where the website may be accessible by a doctor or medical professional. In an embodiment, the patch communicates with the mobile device via Bluetooth or another similar signal transmission method or protocol. In an embodiment, if Bluetooth is unavailable, data may be stored in the internal memory and transmitted to the mobile device when a connection is established.

In an embodiment, the sensors and/or components of the patch collect data in a raw form.

In a further embodiment, the raw data is transmitted to a mobile device, server, and/or website, where one or more locations is configured to convert the raw data to presentable data. In an embodiment, the sensors and/or components of the patch collect generalized information (i.e., the status of the battery, whether the patch is properly interfaced with the skin, or if the patient is resting).

The housing may be composed of a waterproof material or may include a waterproof coating. In an embodiment, the portion of the housing configured to interface with a patient's skin may include a removable film, wherein the removable film conceals an adhesive. The removable film may be configured to protect the adhesive prior to use.

In an embodiment, the patch may be configured for placement upon any thoracic location. The patch components, internal processing aspects, and/or external processing aspects (i.e., processing within the paired smart device) may be calibrated for a thoracic location. However, in such an embodiment, the data may still require normalization. For example, a doctor may place the patch upon position A, wherein the processing chip 530 is calibrated to position A. Accordingly, the normalization procedure may apply one or more constants or modifiers to one or more variables or biometrics. In yet a further embodiment, the system may include one or more constants for each potential position. Such constants may be determined experimentally or may be calculated via modeling.

The interfacing portion of the patch (i.e., the portion of the housing disposed between the PCB and the patient's skin) may be of a particular dimension (i.e., no more than 1 mm). For example, the light-based sensors may provide the most accurate results when disposed near the surface of the silicone. Yet further, the various components of the PCB may be positioned such that each sensor or component is a desired distance from the patient's skin. Additionally, a first number of components may be disposed on the surface of the PCB configured to face the patient's skin and a second number of components may be disposed on the surface of the PCB configured opposite to the patient's skin. For example, sensors for gathering information from the person may be disposed on the bottom surface of the PCB, while components on the top surface of the PCB may include a management chip, an accelerometer 570, a memory, and/or a battery.

The interfacing surface of the patch may be flat. The top portion of the patch may be rounded as to be less obtrusive. However, the silicone (or other suitable material) may comprise a degree of plasticity to conform to curves of the body.

In an embodiment, the paired mobile device may analyze data gathered by the patch. However, in alternate embodiments, the patch may include a processor on board. The patch may include a microprocessor configured to analyze transmitted data to confirm that such outgoing data is structurally sound or otherwise properly formatted. Thus, the microprocessor may monitor the data to observe whether the data has been packaged into a usable form, sent to mobile device, and/or received by the mobile device. The microprocessor may be programmed to determine whether data is valid, for example, by sampling several readings of SpO₂ and selecting the best readings of the packet of readings.

The smart device or paired mobile device may include accessibility to the “app.” The “app” or portal may be secure and may permit access only to professionals. For example, such professionals may have an account registered with the portal, allowing the registered users to interact with data related to their patients. Accordingly, the “app” may display data, including live data or historical data.

The patch of the present disclosure may be a wireless vital sign sensor apparatus comprising a transceiver, a power supply, a plurality of sensors, comprising a heartrate monitor, a gyroscope 580, and an accelerometer 570, a non-transitory memory module capable of storing machine executable code, a processor configured to package data to be transmitted to an external device based on a criteria, determine a clinically significant event based on a determined pathology, and provide an indication when the clinically significant event is detected, and a housing made of a soft flexible material.

Aspects of the present disclosure relate to a wireless vital sign sensor apparatus, further including a plurality of ultrasonic emitters and an ultrasonic receiver. Aspects of the present disclosure relate to a wireless vital sign sensor apparatus, wherein the plurality of ultrasonic emitters are in an array that is electronically steerable to an anatomy to be imaged. Aspects of the present disclosure relate to a wireless vital sign sensor apparatus, wherein the array has a parabolic curve to the ultrasonic emitters.

Aspects of the present disclosure relate to a wireless vital sign sensor apparatus, wherein the ultrasonic receiver is located approximately along a central axis of the array. Aspects of the present disclosure relate to a wireless vital sign sensor apparatus, wherein the apparatus is communicatively connected to a second apparatus where the two apparatuses coordinate the ultrasonic emitters of each apparatus to electronically steer both emitted beams inside a patient thoracic cavity. Aspects of the present disclosure relate to a wireless vital sign sensor apparatus, wherein the housing is made of a silicone.

Aspects of the present disclosure relate to a wireless vital sign sensor apparatus, wherein the housing is impregnated with an antimicrobial agent. Aspects of the present disclosure relate to a wireless vital sign sensor apparatus, wherein the housing is impregnated with an anesthetic agent. Aspects of the present disclosure relate to a wireless vital sign sensor apparatus, wherein the housing is impregnated with a prescribed drug.

Aspects of the present disclosure relate to a wireless vital sign sensor apparatus, wherein the housing comprises a plurality of layers, wherein at least one layer has an acoustic impedance similar to human tissue. Aspects of the present disclosure relate to a wireless vital sign sensor apparatus, wherein the housing is shaped in a way that creates a suction force when pressed against a patient. Aspects of the present disclosure relate to a wireless vital sign sensor apparatus, wherein the housing has an adhesive layer on at least one side.

The invention of the present disclosure may be a method of manufacturing a wireless vital sign sensor apparatus comprising removing a printed circuit board (PCB) from a packaging, placing the PCB into a mold, pouring a premeasured liquid housing into the mold, waiting for the premeasured liquid housing to solidify, and applying the apparatus to a patient.

Aspects of the present disclosure relate to a method of manufacturing the wireless vital sign sensor apparatus, wherein the packaging forms the mold. Aspects of the present disclosure relate to a method of manufacturing the wireless vital sign sensor apparatus, further including uploading a program for a pathology to the apparatus using a user device.

Aspects of the present disclosure relate to a method of manufacturing the wireless vital sign sensor apparatus, further including selecting a stored pathology program on apparatus.

The invention of the present disclosure may be a wireless vital sign sensor apparatus comprising a processor configured to; electronically steer a phased array ultrasound emitter, and communicate with a second apparatus wherein both apparatus coordinate the electronic steering based on overlapping ultrasonic beams.

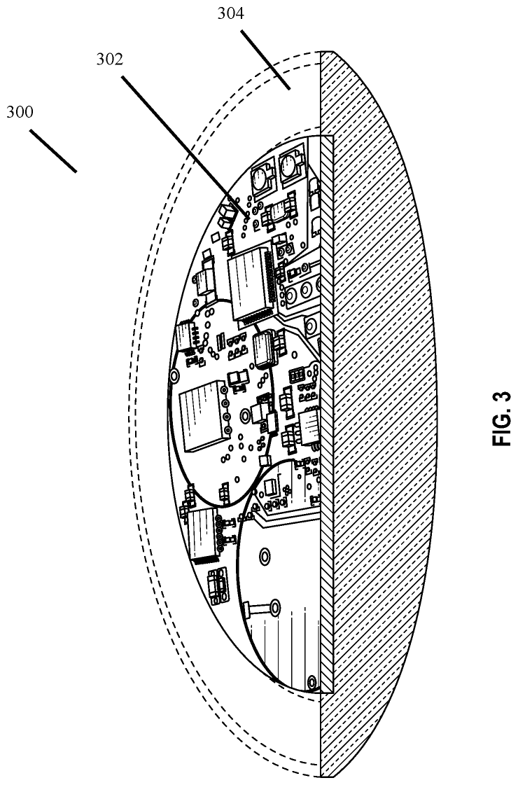

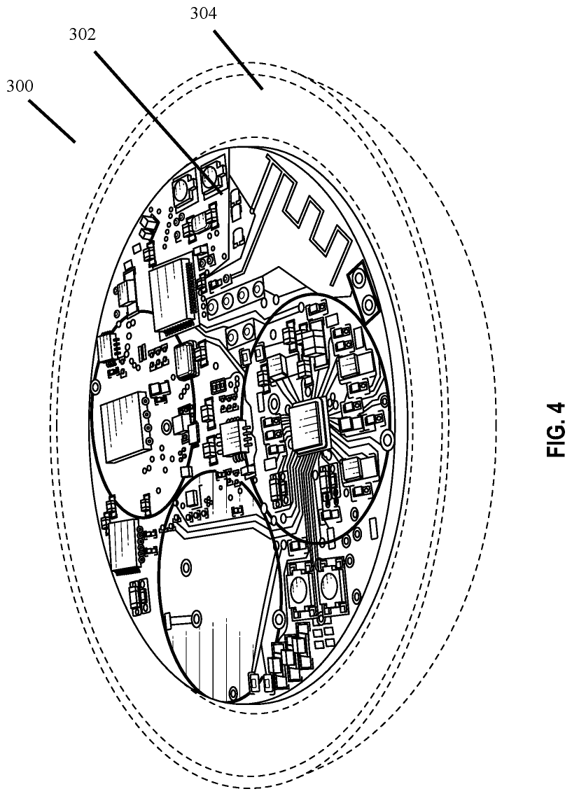

Referring to FIGS. 3-4, the patch 300 may comprise a printed circuit board (“PCB”) 302 encapsulated by a housing 304. For the purposes of this disclosure, the patch 300 may be in wireless communication with the wireless network 110 and/or one or more of the client devices 102-106. Accordingly, the patch 300 may gather data as detailed above and transmit such data to one or more of the client devices 102-106 and/or wireless network 110. The one or more client devices 102-106 may include the “app” and/or portal as described above. Accordingly, the one or more client devices 102-106 may permit data aggregation, manipulation, analysis, and data display to an authenticated user, such as a medical professional.

In an embodiment, the patch 300 is configured to advance the technology of wearable smart devices for aiding the diagnosis and monitoring of health conditions. Thus, the patch 300 may be a complete medical device that is noninvasive and simple to use. In such an embodiment, the patch 300 may be disease-agnostic and comprise capabilities for a full suite of recorded metrics.

In an embodiment, in order to perform a complete health workup, the patch 300 may include numerous interconnected systems (e.g., breath sensing, electrocardiogram (ECG), etc.). Thus, the patch 300 comprises a power source configured for such power consumption, for example, permitting the patch 300 to run for approximately one week while adhered to a user. Accordingly, the entire system may include a low power consumption and a means of wireless communication to an external device. Further, the patch 300 and components thereof may be adapted to transmit data at a standard rate to provide live information.

In an embodiment, the chip may require an external method with which to toggle the power. Specifically, the patch 300 may be fully encased in silicone such that it is waterproof and could adhere to the body. In such an embodiment, no form of on/off capability is possible local to the device. Moreover, a Bluetooth chip may be required to enable the device to communicate with a phone and then to a server. Finally, some onboard memory may be required to store local data before it could be passed to an external device.

In an embodiment, development boards and breadboard test circuits may be combined with a Bluetooth module, microprocessor, and/or some of the health components (e.g., heart rate and blood oxygen monitoring). In a different embodiment, an Arduino Bluno Bluetooth module, Maxim evaluation kits for each component, and a Nordic microcontroller may be utilized, such that the test board may successfully communicate with a mobile device. A plurality of components (e.g., an accelerometer, a temperature sensor, an ECG readout chip, a microphone, and/or a near-field communication chip) may be used to at least one of: measure body position, measure temperature, take ECG readouts, analyze breathing, and activate the device. In a further embodiment, said plurality of components may enable the device to analyze the heart rate (beats/minute) of an individual through an ECG, analyze their breathing using a microphone situated on the user's chest, and determine an individual's blood oxygen saturation, body temperature, and motion through the wearable smart patch.

Each measurement component may be built on a different evaluation kit, which may ensure adequate functionality and power requirements. I2C communication may be used for the Bluetooth hardware to remain viable. Each component may be assigned to its own memory mapping, which in turn, may store information. Individual connection pins may be used to modify or complete a firmware update.

A software may have an I2C interface (or similar module) within it that may allow alteration of pin information and communication methods. The system may generate a clock and may monitor the rate with an oscilloscope until the clock reaches the same rate as the chip. Further, the software for each pin may be altered to hardcode whether the information was input or output. This step may be used for both testing and integration purposes. In a further embodiment, the system may include a function within the software that may write the component information directly to the microprocessor memory. Accordingly, all components and pins may be temporarily modified to a higher frame rate and the firmware update may be ubiquitously applied. With the firmware updated and proprietary software in place, all chips and components may be returned to their base states to ensure low power consumption and correct functionality for the medical use-case.

In an embodiment, the firmware may be adapted to control the custom software, access stored memory, and/or communicate effectively with an external device. The system may also include a calibration protocol to determine the position of a user's body and/or analyze different activities/stages of sleep. As a non-limiting example, the patch may provide a solution that can perform efficiently within the given constraints.

In an embodiment, a wearable smart patch may monitor an individual's vitals. Specifically, low-power requirements may mean that health monitoring devices may be efficient in their communication protocols and conserve memory where possible. This may be utilized for the medical application to enable a sufficiently long wear time. A custom software and firmware may permit such device functionality. In an embodiment, software libraries may read-out sensor information into a single, accessible data table.

While this invention has been described in conjunction with the embodiments outlined above, many alternatives, modifications and variations will be apparent to those skilled in the art upon reading the foregoing disclosure. Accordingly, the embodiments of the invention, as set forth above, are intended to be illustrative, not limiting. Various changes may be made without departing from the spirit and scope of the invention.

What is claimed is:

1. A wireless vital sign patch apparatus comprising:
a printed circuit board (“PCB”) comprising:
a transmitter;
a plurality of sensors comprising a heartrate monitor, a gyroscope, and an accelerometer;
at least one processor, at least one memory comprising computer-executable instructions which, when executed by the at least one processor, cause the at least one processor to:
package data to be transmitted, via the transmitter, to an external device based on a criteria; and
a housing encapsulating the PCB, the housing composed a flexible material, the housing comprising an interfacing surface configured to reversibly interface with a patient.

2. The apparatus of claim 1, wherein the flexible material is transparent.

3. The apparatus of claim 1, wherein the flexible material is silicone.

4. The apparatus of claim 1, wherein the housing is impregnated with an antimicrobial agent.

5. The apparatus of claim 1, wherein the housing is impregnated with an anesthetic agent.

6. The apparatus of claim 1, wherein the housing is impregnated with a prescribed drug.

7. The apparatus of claim 1, the housing further comprising an adhesive disposed on the interfacing surface.

8. The apparatus of claim 1, wherein the flexible material includes an acoustic impedance similar to that of human tissue.

9. A wireless vital sign detection system comprising:
a patch comprising:
a printed circuit board (“PCB”) comprising:
a transmitter;
a plurality of sensors comprising a heartrate monitor, a gyroscope, and an accelerometer;
at least one patch processor, at least one patch memory comprising computer-executable patch instructions which, when executed by the at least one patch processor, cause the at least one patch processor to:
package data to be transmitted, via the transmitter, to an external device based on a criteria;
a housing encapsulating the PCB, the housing composed a flexible material, the housing comprising an interfacing surface configured to reversibly interface with a patient; and
an external device comprising:
a receiver in wireless communication with the transmitter;
at least one external processor, at least one external memory comprising computer-executable external instructions which, when executed by the at least one external processor, cause the at least one external processor to:
receive, via the receiver, the packaged data;
determine, via the at least one external processor, a clinically significant event based on a determined pathology; and
provide an indication when the clinically significant event is detected.

10. The system of claim 9, wherein the flexible material is silicone.

11. The system of claim 9, wherein the housing is impregnated with an antimicrobial agent.

12. The system of claim 9, wherein the housing is impregnated with an anesthetic agent.

13. The system of claim 9, wherein the housing is impregnated with a prescribed drug.

14. The system of claim 9, the housing further comprising an adhesive disposed on the interfacing surface.

15. The system of claim 9, wherein the flexible material includes an acoustic impedance similar to that of human tissue.

16. The system of claim 1, wherein the flexible material is transparent.
